(19) United States
(12) Patent Application Publication
(10) Pub. No.: US 2023/0101403 A1
(43) Pub. Date: Mar. 30, 2023

(54) METHOD OF TREATING OR AMELIORATING CANCERS DRIVEN BY RECEPTOR TYROSINE KINASE FUSION ONCOGENES, AND COMPOSITIONS FOR THE SAME

(71) Applicant: THE TRUSTEES OF THE UNIVERSITY OF PENNSYLVANIA, Philadelphia, PA (US)

(72) Inventors: Lukasz Jan Bugaj, Philadelphia, PA (US); David Gonzalez-Martinez, Philadelphia, PA (US)

(21) Appl. No.: 17/936,654

(22) Filed: Sep. 29, 2022

Related U.S. Application Data
(60) Provisional application No. 63/250,083, filed on Sep. 29, 2021.

(51) Int. Cl. A61K 38/00; A61P 35/00

(52) U.S. Cl. CPC A61K 38/005; A61P 35/00

(57) ABSTRACT

Described herein is a method of treating or ameliorating cancer in a subject in need thereof. The cancer includes a cytoplasmic RTK fusion protein aggregate. The method includes administering to the subject an inhibitor for the RTK fusion protein, and an inhibitor for matrix metalloprotease. Also described herein is a composition or a kit for treating or ameliorating cancer according to the method.

### CROSS-REFERENCE TO RELATED APPLICATIONS

The present application claims priority under 35 U.S.C. § 119(e) to U.S. Provisional Patent Application No. 63/250,083 filed Sep. 29, 2021, which is incorporated herein by reference in its entirety.

### STATEMENT REGARDING FEDERALLY SPONSORED RESEARCH

This invention was made with government support under GM138211 awarded by the National Institutes of Health. The government has certain rights in the invention.

### BACKGROUND

Receptor tyrosine kinase (RTK) fusion oncogenes are drivers for many types of cancers. In cancers driven by RTK fusions, inhibitors of the oncogenic RTK sometimes successfully block signaling and lead to initial tumor remission. However, resistance to the treatment ultimately emerges in most of the cases.

Therefore, there is a need to develop novel methods and compositions that can provide durable treatment for cancers driven by RTK fusion oncogenes. The instant invention addresses this need.

### SUMMARY

In some aspects, the present invention is directed to the following non-limiting embodiments:

### Method of Treating or Ameliorating Cancer

In some aspects, the present invention is directed to a method of treating or ameliorating a cancer in a subject in need thereof.

In some embodiments, the method includes administering to the subject:
an effective amount of an inhibitor for the receptor tyrosine kinase (RTK) fusion protein; and
an effective amount of an inhibitor for a matrix metalloprotease (MMP).

In some embodiments, a tumor cell in the cancer includes an aggregate of the RTK fusion protein.

In some embodiments, the RTK fusion protein is at least one selected from the group consisting of an anaplastic lymphoma kinase (ALK) fusion protein, a rearranged during transfection (RET) fusion protein, a ROS1 fusion protein, and a neurotrophic receptor tyrosine kinase 1 (NTRK1) fusion protein.

In some embodiments, the RTK fusion protein is at least one selected from the group consisting of EML4-ALK, NPM-ALK, CCDC6-RET, KIF5B-RET, NCOA-RET, TRIM33-RET, CUX1-RET, and KIAA1468-RET.

In some embodiments, the cytoplasmic granule of the RTK fusion protein sequesters an effector of a transmembrane RTK signaling pathway from the transmembrane RTK signaling pathway.

In some embodiments, the cytoplasmic granule of the RTK fusion protein sequesters growth factor receptor bound protein 2 (GRB2) from a transmembrane RTK signaling pathway.

In some embodiments, the RTK fusion protein includes an ALK fusion protein, and the inhibitor for the RTK fusion protein includes at least one ALK inhibitor selected from the group consisting of Alectinib, Alkotinib (also known as ZG-0418), AP26113, ASP3026, AZD3463, Belizatinib (also known as TSR-011), Brigatinib, CEP-28122, CEP-37440, Certinib, Crizotinib, Ensartinib (also known as X-396), Entrectinib (also known as NMS-E628 and RXDX-101), Foritinib (SAF-189), HG-14-10-04, Lorlatinib, PF-06463922, PLB1003, Repotrectinib (also known as TPX-0005), TAE684, TPX-0131, TQ-B3139, TSR-011, X-376, and a derivative thereof.

In some embodiments, the RTK fusion protein includes an RET fusion protein, and the inhibitor for the RTK fusion protein includes at least one selected from the group consisting of alectinib, cabozantinib, lenvatinib, pralsetinib, selpercatinib, sunitinib, and a derivative thereof.

In some embodiments, the MMP includes at least one selected from the group consisting of ADAM 10 and ADAM 17.

In some embodiments, the inhibitor for MMP includes at least one selected from the group consisting of 1,10-Phenanthroline Monohydrate, Abametapir, Aderbasib, AUDA, Auraptene, Batimastat, Cipemastat, Doxycycline Hyclate, GI254023X, Ilomastat, Lactobionic acid, Ilomastat (Galardin), JNJ0966, Marimastat, MMI270, MMP-9-IN-1, Morroniside, Nobiletin, NSC 405020, o-phenanthroline, Polygalactic acid, Prinomastat, Rebimastat, Ro 28-2653, RS-130830, SB-3CT, Solasodine, Tanomastat, TAPI-1, Trans-Zeatin, Triolein, T-5224, T-26c, and a derivative thereof.

In some embodiments, the subject is a human.

### Pharmaceutical Composition for Treating or Ameliorating Cancer

In some aspects, the present invention is directed to a pharmaceutical composition for treating or ameliorating a cancer in a subject in need thereof.

In some embodiments, the composition includes:
an inhibitor for the RTK fusion protein;
an inhibitor for a matrix metalloprotease (MMP); and
a pharmaceutically acceptable carrier.

In some embodiments, the inhibitor for the RTK fusion protein, the inhibitor for MMP and the pharmaceutically acceptable carrier are co-formulated.

In some embodiments, a tumor cell in the cancer inlcudes an aggregate of a receptor tyrosine kinase (RTK) fusion protein.

In some embodiments, the RTK fusion protein is at least one selected from the group consisting of an anaplastic lymphoma kinase (ALK) fusion protein, a rearranged during transfection (RET) fusion protein, a ROS1 fusion protein, and a neurotrophic receptor tyrosine kinase 1 (NTRK1) fusion protein.

In some embodiments, the RTK fusion protein is at least one selected from the group consisting of EML4-ALK, NPM-ALK, CCDC6-RET, KIFSB-RET, NCOA-RET, TRIM33-RET, CUX1-RET, and KIAA1468-RET.

In some embodiments, the cytoplasmic granule of the RTK fusion protein sequesters an effector of a transmembrane RTK signaling pathway from the transmembrane RTK signaling pathway.

In some embodiments, the cytoplasmic granule of the RTK fusion protein sequesters growth factor receptor bound protein 2 (GRB2) from a transmembrane RTK signaling pathway.

In some embodiments, the RTK fusion protein includes an ALK fusion protein, and the inhibitor for the RTK fusion protein includes at least one ALK inhibitor selected from the group consisting of Alectinib, Alkotinib (also known as ZG-0418), AP26113, ASP3026, AZD3463, Belizatinib (also known as TSR-011), Brigatinib, CEP-28122, CEP-37440, Certinib, Crizotinib, Ensartinib (also known as X-396), Entrectinib (also known as NMS-E628 and RXDX-101), Foritinib (SAF-189), HG-14-10-04, Lorlatinib, PF-06463922, PLB1003, Repotrectinib (also known as TPX-0005), TAE684, TPX-0131, TQ-B3139, TSR-011, X-376, and a derivative thereof.

In some embodiments, the RTK fusion protein includes an RET fusion protein, and the inhibitor for the RTK fusion protein includes at least one selected from the group consisting of alectinib, cabozantinib, lenvatinib, pralsetinib, selpercatinib, sunitinib, and a derivative thereof.

In some embodiments, the MMP includes at least one selected from the group consisting of ADAM 10 and ADAM 17.

In some embodiments, the inhibitor for MMP includes at least one selected from the group consisting of 1,10-Phenanthroline Monohydrate, Abametapir, AUDA, Auraptene, Batimastat, Cipemastat, Doxycycline Hyclate, GI254023X, Ilomastat, Lactobionic acid, Ilomastat (Galardin), JNJ0966, Marimastat, MMI270, MMP-9-IN-1, Morroniside, Nobiletin, NSC 405020, o-phenanthroline, Polygalactic acid, Prinomastat, Rebimastat, Ro 28-2653, RS-130830, SB-3CT, Solasodine, Tanomastat, TAPI-1, Trans-Zeatin, Triolein, T-5224, T-26c, and a derivative thereof.

In some embodiments, the subject is a human.

### Kit for Treating or Ameliorating Cancer

In some aspects, the present invention is directed to a kit for treating or ameliorating a cancer in a subject in need thereof.

In some embodiments, the kit includes:
an inhibitor for the RTK fusion protein;
an inhibitor for a matrix metalloprotease (MMP).

In some embodiments, the kit further includes one or more pharmaceutically acceptable carriers.

In some embodiments, the inhibitor for the RTK fusion protein is co-formulated with a first pharmaceutically acceptable carrier. In some embodiments, the inhibitor for MMP is co-formulated with a second pharmaceutically acceptable carrier.

In some embodiments, a tumor cell in the cancer includes an aggregate of a receptor tyrosine kinase (RTK) fusion protein.

In some embodiments, the RTK fusion protein is at least one selected from the group consisting of an anaplastic lymphoma kinase (ALK) fusion protein, a rearranged during transfection (RET) fusion protein, a ROS1 fusion protein, and a neurotrophic receptor tyrosine kinase 1 (NTRK1) fusion protein.

In some embodiments, the RTK fusion protein is at least one selected from the group consisting of EML4-ALK, NPM-ALK, CCDC6-RET, KIF5B-RET, NCOA-RET, TRIM33-RET, CUX1-RET, and KIAA1468-RET.

In some embodiments, the cytoplasmic granule of the RTK fusion protein sequesters an effector of a transmembrane RTK signaling pathway from the transmembrane RTK signaling pathway.

In some embodiments, the cytoplasmic granule of the RTK fusion protein sequesters growth factor receptor bound protein 2 (GRB2) from a transmembrane RTK signaling pathway.

In some embodiments, the RTK fusion protein includes an ALK fusion protein, and the inhibitor for the RTK fusion protein includes at least one ALK inhibitor selected from the group consisting of Alectinib, Alkotinib (also known as ZG-0418), AP26113, ASP3026, AZD3463, Belizatinib (also known as TSR-011), Brigatinib, CEP-28122, CEP-37440, Certinib, Crizotinib, Ensartinib (also known as X-396), Entrectinib (also known as NMS-E628 and RXDX-101), Foritinib (SAF-189), HG-14-10-04, Lorlatinib, PF-06463922, PLB1003, Repotrectinib (also known as TPX-0005), TAE684, TPX-0131, TQ-B3139, TSR-011, X-376, and a derivative thereof.

In some embodiments, the RTK fusion protein includes an RET fusion protein, and the inhibitor for the RTK fusion protein includes at least one selected from the group consisting of alectinib, cabozantinib, lenvatinib, pralsetinib, selpercatinib, sunitinib, and a derivative thereof.

In some embodiments, the MMP includes at least one selected from the group consisting of ADAM 10 and ADAM 17.

In some embodiments, the inhibitor for MMP includes at least one selected from the group consisting of 1,10-Phenanthroline Monohydrate, Abametapir, Aderbasib, AUDA, Auraptene, Batimastat, Cipemastat, Doxycycline Hyclate, GI254023X, Ilomastat, Lactobionic acid, Ilomastat (Galardin), JNJ0966, Marimastat, MMI270, MMP-9-IN-1, Morroniside, Nobiletin, NSC 405020, o-phenanthroline, Polygalactic acid, Prinomastat, Rebimastat, Ro 28-2653, RS-130830, SB-3CT, Solasodine, Tanomastat, TAPI-1, Trans-Zeatin, Triolein, T-5224, T-26c, and a derivative thereof.

In some embodiments, the subject is a human.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood; however, that the instant specification is not limited to the precise arrangements and instrumentalities of the embodiments shown in the drawings.

FIGS. 1A-1F demonstrate that RTK signaling is suppressed in EML4-ALK+ cancer cells, in accordance with some embodiments. FIG. 1A illustrates that EML4-ALK+ cancer cells treated with ALK specific tyrosine kinase inhibitor (TKI) often result in a population of persistent/tolerant cells that later develop into resistant colonies. FIG. 1B shows ALK immunostaining results of EML4-ALK+ cancer cell lines STE-1 and H3122, which shows aggregates of EML4-ALK. FIG. 1C, left panel, illustrates that transmembrane RTK and EML4-ALK protein granules both signal through the same adapter proteins and downstream pathways like Ras/Erk. FIG. 1C, right panel, illustrates the functional profiling method used in determining the extent to which EML4-ALK expression or its inhibition alter signal transmission in cancer cells. FIG. 1D illustrates that optoFGFR allows for blue light stimulation of RTK (optoFGFR) signal transduction. FIG. 1E depicts the single-cell analysis of ppErk levels in STE-1 cells stimulated with light (optoFGFR) in the presence of either DMSO (upper panel) or 1μM Crizotinib (lower panel). FIG. 1F depicts the normalized ppErk fold-change in response to 5 min of blue light stimulation. Blue light intensity ranged from 2% to 60% of maximum (1.45 W/cm\hat{ }2). Datapoints show mean single-cell ppErk intensity from three replicates.

FIGS. 2A-2F demonstrate that EML4-ALK suppresses—and ALK inhibition restores—EGFR signaling, in accordance with some embodiments. FIG. 2A illustrates the activation of EGFR signal transduction using epidermal growth factor (EGF). FIG. 2B depicts the ppErk levels in response to 100 ng/mL EGF stimulation in the presence of either DMSO or 1 μM crizotinib in both STE-1 and H3122 cancer cells. Data points represent mean single cell ppErk intensity of three replicates. FIG. 2C depicts the quantification of dynamic range of ppErk induction from experiment in 2C performed over a range of EGF concentrations. FIG. 2D illustrates the transient transfection of EML4-ALK(V1)-2A-H2B-iRFP in Beas2B cells. FIG. 2E depicts the ppErk immunofluoresence intensity response to 50 ng/mL EGF stimulation. Cells were analyzed as a function of miRFP expression. FIG. 2F depicts the dynamic range of ppErk induction in Beas2B cells transiently expressing EML4-ALK and treated with DMSO or 1 μM crizotinib before stimulation.

FIGS. 3A-3L demonstrate that EML4-ALK aggregates suppress EGFR through sequestration of RTK effectors, in accordance with some embodiments. FIG. 3A shows that STE-1 cells were preincubated with 1 μM crizoitinib at varying times before stimulation with 50 ng/mL EGF for 15 min followed by fixation and ppErk immunostaining. FIG. 3B depicts the ppErk immunostaining intensity after 15 min of 50 ng/mL EGF stimulation following varying durations of pre-incubation with 1 μM crizotinib. FIG. 3C illustrates that Beas2B Grb2::mNG were transiently transfected with EML4-ALK-2A-H2B-miRFP and stimulated with 50 ng/mL EGF to visualize Grb2 translocation in the presence and absence of EML4-ALK. FIG. 3D shows the live cell imaging of Grb2 translocation in response to EGF in the presence and absence of EML4-ALK expression. FIG. 3E depicts the line scan of intensity of Grb2 fluorescence distribution after EGF stimulation in the absence (“wt”) or presence (“+EML4-ALK”) of EML4-ALK expression. FIG. 3F is the schematic for visualization of Grb2 localization upon treatment with 1 μM crizotinib. FIG. 3G shows the fluorescence imaging of Grb2 localization upon crizotinib treatment. FIG. 3H depicts the quantification of dissociaton of Grb2 puncta upon crizotinib treatment in EML4-ALK-expressing cells. FIG. 3I is a schematic of visualization of Grb2 distribution upon sequential treatment with crizotinib and EGF. FIG. 3J shows the fluorescence imaging of Grb2 distribution upon sequential treatment with crizotinib and EGF. FIG. 3K depicts the ppErk induction in response to EGF stimulation in STE-1 cells that overexpress Grb2-GFP. FIG. 3L shows a model according to the present study. According to the model, active cytoplasmic EML4-ALK protein granules inhibitor RTK signals through sequestration of the Grb2 signaling effector. Treatment with ALK inhibitor releases sequestered Grb2 and potentiates RTK signals.

FIGS. 4A-4I and FIGS. 5A-5D demonstrate RTK hypersensitization upon ALK inhibition results in rapid signal reactivation through paracrine signaling, in accordance with some embodiments. FIG. 4A illustrates that ErkKTR was stably integrated into STE-1 cells for live tracking of ppErk signal activity in response to ALKi treatment. FIG. 4B shows live cell imaging of STE-1 cells stably expressing ErkKTR treated with DMSO or 1 μM crizotinib. FIG. 4C depicts the single-cell traces of cytoplasmic/nuclear ErkKTR intensity ratio in STE-1 cells treated with DMSO or 1 μM crizotinib. FIG. 4D shows the heatmaps of ErkKTR activity in STE-1 cells treated with DMSO, 1 μM crizotinib, 1 μM crizotinib+1 μM erlotinib, 1 μM crizotinib+10 μM marimastat. FIG. 4E depicts the count of Erk activation pulses in STE-1 treated with DMSO, crizotinib(“ALKi”), crizotinib+erlotinib, or crizotinib+marimastat (“ALKi MMPi”). FIG. 4F depicts the percentage of STE-1 cells exhibiting more than 1 pulse following inhibitor treatment. Error bars represent 95% confidence intervals. FIG. 4G illustrates that RTK/Erk activation stimulates transcription of immediate early genes including EGR1. FIG. 4H depicts the EGR1 expression in individual STE-1 cells following treatment with 1 μM crizotinib, 1 μM crizotinib+1 μM erlotinib, 1 μM crizotinib+50 ng/mL EGF. FIG. 4I depicts the comparison of EGR1 expression after 6 hr of inhibitor treatment. FIG. 5A shows live cell imaging of ErkKTR activity in proximity to dying cells in STE-1 cells treated with the indicated drugs. FIG. 5B is the schematic of spatial analysis of Erk signaling in relation to dying cells. Cells within a 32 μm radius of a dying cell were considered neighbors of the dying cell, whereas all other cells were non-neighbors. Neighbors/non-neighbors were identified and tracked for 50 min before the death event, and Erk actvity in these cells was quantified. FIG. 5C depicts the quantification of Erk activity pulses in neighbors (“N”) or non-neighbors (“R”) of each cell-death event in the indicated conditions. FIG. 5D depicts the percentage of neighbor and non-neighbor STE-1 cells exhibiting more than 1 pulse in response to cell death events upon inhibitor treatments.

FIGS. 6A-6G demonstrate that signal reactivation promotes acute drug tolerance and cell persistence in the presence of ALK inhibition, in accordance with some embodiments. FIG. 6A shows that, although ALK inhibition promotes cell death of EML4-ALK+ cells, paracrine signals may promote drug tolerance and survival. FIG. 6B shows the heatmap of Erk activity and cell death (red dots) in a random sample of cells from each condition. FIG. 6C depicts the quantification of death in STE-1 cells following 24 hrs of inhibitor treatment. FIG. 6D depicts the number of Erk pulses in cells that died (“D”) and cells that lived (“L”) during 24 hrs of inhibitor treatment. FIG. 6E depicts the percentage of caspase-3+ STE-1 and H3122 cells following 24 hrs of inhibitor treatment.

FIG. 7 demonstrates that the second generation ALK inhibitor, Alectinib, increases Caspase-3 activation in STE-1 cancer cells when combined with MMP inhibitor, in accordance with some embodiments. Percent of Caspase-3 positive cells assessed using NucView 488 afterfive days of treatment with the indicated inhibitors: Alecitinib 1 μM, Erlotinib 1 μM, Marimastat 10 μM.

FIG. 8 demonstrates that the tolerance of cancer H3122 cells to ALK inhibitor drug decreases with MMP inhibition, in accordance with some embodiments. Crystal violet staining (left panels) and cell counts (right panel) of cell survival after 17 days of the indicated treatments: Crizotinib 1 μM, Erlotinib 1 μM, Marimastat 10 μM.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Receptor tyrosine kinase (RTK) fusion genes are a large class of oncogenes, which are formed by translocation and other chromosomal rearrangements of a subset of receptor tyrosine kinase genes. An RTK fusion gene includes a segment from the tyrosine kinase gene, which normally includes the kinase domain, as well as a fragment from a partner gene.

Many oncogenic RTK fusion proteins form higher-order assemblies, such as in form of protein aggregates or granules (the terms “aggregate” and “granule” are used interchangeably herein), in tumor cells. The protein aggregates are formed due to that the partner protein of these oncogenic RTK fusion proteins oligomerize, and that RTK fusion proteins further bind to scaffold proteins of RTK pathways, some of which interact with the RTK fusions in a multivalent manner. Depending on the natures of the RTK fusions (e.g., whether the partner proteins include transmembrane domains), the RTK fusion aggregates are formed on the plasma membrane, or in the cytoplasm. The higher-order assemblies of the oncogenic fusion proteins locally concentrate, among others, effector proteins of RTK signaling pathways. For example, cytoplasmic aggregates or granules of ALK fusion proteins or RET fusion proteins lead to activation of downstream MAPK signaling and therefore oncogenesis. (Tulpule et al., Cell. 13;184(10):2649-2664 (2021), the entirety of the reference is hereby incorporated herein by reference).

EML4-ALK, which drives ˜3-7% of non-small cell lung cancer (NSCLC), is one of such RTK fusion oncogenes. The fusion protein of EML4-ALK includes the kinase domain of ALK, as well as a fragment from echinoderm microtubule-associated protein-like 4 (EML4). The EML4-ALK fusion protein aggregates and forms large protein granules in the cytoplasm of tumor cells. The aggregation of EML4-ALK fusion protein is caused by the oligomerization of the EML4-ALK fusion protein through the EML4 portion, and amplified by RTK adapter proteins like Grb2, which serve as scaffolds that attach to multiple EML4-ALK oligomers through multivalent interactions.

ALK inhibitors, such as crizotinib, are effective for treating NSCLC harboring chromosomal rearrangements of ALK. However, resistances to ALK inhibitors ultimately develops in the patients after prolonged treatment.

The study described herein (“the present study”), using EML4-ALK driven cancer cell lines as a non-limiting illustrative example, demonstrates that, in the absence of RTK inhibition, the high-order assemblies (e.g., cytoplasmic granules or aggregates, or membrane aggregates) of oncogenic RTK fusion protein in the tumor cells dramatically suppress signaling through the transmembrane RTKs by sequestration of downstream effectors for the transmembrane RTKs. However, inhibition of the oncogenic RTK fusion proteins by inhibitors potentiates signaling through the transmembrane RTKs beyond levels achievable in the absence of RTK fusion protein inhibition. The potentiated transmembrane RTK signaling promotes cell survival by compensation of the suppressed survival signals by the oncogenic RTK fusions, which at least partially explains the drug resistance. The present study further illustrates that combining the inhibition of the RTK fusion protein with inhibition of the transmembrane RTK signaling results in a significantly higher percentage of cell death in comparison to treating cells with only the inhibitor for the RTK fusion protein, or to treating cells with only the inhibitors of transmembrane RTK signaling. Examples of such inhibitors of transmembrane RTK signaling include inhibitors for matrix metalloproteases (MMPs).

Accordingly, in some aspects, the instant specification is directed to a method of treating or ameliorating a cancer having an RTK fusion protein aggregation in a subject in need thereof. The method includes administering to the subject: an effective amount of an inhibitor for the RTK fusion protein; and an effective amount of an inhibitor for a matrix metalloprotease.

In some aspects, the present invention includes a composition for treating or ameliorating a cancer having an RTK fusion protein aggregation in a subject in need thereof. The composition includes an inhibitor for the RTK fusion protein; and an inhibitor for a matrix metalloprotease.

It is worth noting that, although the present study uses EML4-ALK+ cancer cells as an illustrative example, the scope of the instant specification is not limited thereto. One of ordinary skill in the art would understand that many RTK fusions share the same features that renders EML4-ALK+ cancer cells susceptible to the methods and compositions described herein, such as the ability to form high-order protein assemblies and to sequester RTK effectors from the membrane. One of ordinary skill in the art would also understand that the instant methods and compositions are expected to be effective in treating or ameliorating other types of cancers that are driven by similar RTK fusions. Non-limiting examples of RTK fusions include other ALK fusions such as NPM-ALK; RET fusions such as CCDC6-RET, KIF5B-RET, NCOA-RET, TRIM33-RET, CUX1-RET, KIAA1468-RET; ROS1 fusions such as TPM3-ROS1, FIG-ROS1, and CCDC6-ROS1; NTRK1 fusions such as MPRIP-NTRK1; and the like.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

In one aspect, the terms “co-administered” and “co-administration” as relating to a subject refer to administering to the subject a compound and/or composition of the disclosure along with a compound and/or composition that may also treat or prevent a disease or disorder contemplated herein. In certain embodiments, the co-administered compounds and/or compositions are administered separately, or in any kind of combination as part of a single therapeutic approach. The co-administered compounds and/or compositions may be administered together, or sequentially in any order. The co-administered compound and/or composition may be formulated in any kind of combinations as mixtures of solids and liquids under a variety of solid, gel, and liquid formulations, and as a solution.

As used herein, the term “pharmaceutical composition” or “composition” refers to a mixture of at least one compound useful within the disclosure with a pharmaceutically acceptable carrier. The pharmaceutical composition facilitates administration of the compound to a patient. Multiple techniques of administering a compound exist in the art including, but not limited to, subcutaneous, intravenous, oral, aerosol, inhalational, rectal, vaginal, transdermal, intranasal, buccal, sublingual, parenteral, intrathecal, intragastrical, ophthalmic, pulmonary, and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

As used herein, the term “treatment” or “treating” is defined as the application or administration of a therapeutic agent, i.e., a compound useful within the disclosure (alone or in combination with another pharmaceutical agent), to a patient, or application or administration of a therapeutic agent to an isolated tissue or cell line from a patient (e.g., for diagnosis or ex vivo applications), who has a disease or disorder and/or a symptom of a disease or disorder, with the purpose to cure, heal, alleviate, relieve, alter, remedy, ameliorate, improve or affect the disease or disorder and/or the symptoms of the disease or disorder. Such treatments may be specifically tailored or modified, based on knowledge obtained from the field of pharmacogenomics.

### Method of Treating or Ameliorate Cancer

As detailed elsewhere herein, the present study establishes that the sequestration of transmembrane RTK signaling pathway effectors, such as Grb2 or SOS proteins (such as SOS1), by an aggregate of an exemplary oncogenic RTK fusion protein (EML4-ALK, which forms a cytoplasmic aggregate) in tumor cells, prevents the effectors from translocating to the membrane to transduce EGF signals. This results in competitive inhibition of the transmembrane RTK signaling. Inhibition of the oncogenic RTK fusion protein alone releases the effectors from the sequestration, thereby re-activating the transmembrane RTK signaling and providing a survival signal in alternative to the ones mediated by the oncogenic RTK fusion protein. This alternative survival signal (at least partially) explains drug resistances that RTK fusion oncogene driven cancers ultimately develop after prolonged treatment with an RTK fusion protein inhibitor.

The present study further establishes that the combined inhibition of the RTK fusion protein (with an inhibitor for the RTK) and the transmembrane RTK signaling pathway (with an inhibitor for MMPs, which cleaves extracellular portions of some transmembrane proteins to produce extracellular EGF signals) achieves a significantly higher death rate in a cancer cell line driven by the RTK fusion oncogene than that achievable by inhibiting the RTK fusion protein alone. Inhibiting the transmembrane RTK signaling pathway alone with the inhibitor for MMP did not result in detectable cell death in the cancer cell line.

As such, the results of the present study illustrate the unexpected results of combining the inhibition of RTK fusion protein with the inhibition of MMP protein.

Accordingly, in some aspects, the instant specification is directed to a method of treating or ameliorating a cancer in a subject in need thereof. The cancer includes an aggregation of a receptor tyrosine kinase (RTK) fusion protein. The method includes administering to the subject an effective amount of an inhibitor for the RTK fusion protein, and an effective amount of an inhibitor for a matrix metalloprotease (MMP).

In some embodiments, the inhibitor for the RTK fusion protein or the inhibitor for MMP is administered with a pharmaceutically acceptable carrier to the subject. The pharmaceutically acceptable carrier is detailed elsewhere herein.

In some embodiments, the aggregation of the RTK fusion protein sequesters an effector of transmembrane RTK signaling pathway from the transmembrane RTK signaling pathway. In some embodiments, the effector of the transmembrane RTK signaling pathway includes GRB2 or an SOS protein (such as SOS1).

In some embodiments, the RTK fusion protein includes all RTK fusion proteins that form aggregates, membrane or cytoplasmic, in tumor cells. In some embodiments, the RTK fusion protein includes ALK fusions such as a EML4-ALK fusion (including variants such as v1, v2, v3a/b, v4, v5a/b, v5′, v7, v8a/b, which are described in Zhang et al., Lung Cancer. 2021 August;158:126-136), or NPM-ALK; RET fusions such as CCDC6-RET, KIF5B-RET, NCOA-RET, TRIM33-RET, CUX1-RET, or KIAA1468-RET; ROS1 fusions such as TPM3-ROS1, FIG-ROS1, CCDC6-ROS1, or CD74-ROS1; NTRK1 fusions such as MPRIP-NTRK1 or TPM3-NTRK1; and the like. In some embodiments, the aggregation of the RTK fusion protein is a cytoplasmic protein granule. In some embodiments, the RTK fusion protein is a fusion protein including the kinase domain of anaplastic lymphoma kinase (ALK) or the kinase domain of rearranged during transfection (RET). In some embodiments, the fusion protein includes a fusion partner capable of multimerization. Both ALK fusion proteins and RET fusion proteins are known to form cytoplasmic protein granules in tumor cells that include effectors of transmembrane RTKs, such as GRB2 or an SOS protein (such as SOS1). As such, it is expected that the effector sequestration as well as the reactivation of transmembrane RTK signaling that causes drug resistance upon inhibiting RTK fusion proteins demonstrated for EML4-ALK is applicable to other ALK fusion proteins and RET fusion proteins. In some embodiments, the fusion protein is at least one selected from the group consisting of EML4-ALK, NPM-ALK, CCDC6-RET, KIF5B-RET, NCOA-RET, TRIM33-RET, CUX1-RET, and KIAA1468-RET.

In some embodiments, the RTK fusion protein is an ALK fusion protein. In some embodiments, the RTK fusion protein is EML4-ALK, NPM-ALK, or a combination thereof. In some embodiments, the inhibitor for the RTK fusion protein is an ALK inhibitor. Non-limiting examples of ALK inhibitors include Alectinib, Alkotinib (also known as ZG-0418), AP26113, ASP3026, AZD3463, Belizatinib (also known as TSR-011), Brigatinib, CEP-28122, CEP-37440, Ceritinib, Crizotinib, Ensartinib (also known as X-396), Entrectinib (also known as NMS-E628 and RXDX-101), Foritinib (SAF-189), HG-14-10-04, Lorlatinib, PF-06463922, PLB1003, Repotrectinib (also known as TPX-0005), TAE684, TPX-0131, TQ-B3139, TSR-011, X-376, or derivatives thereof. ALK inhibitors are well known and three generations of ALK inhibitors are publicly available. As such, one of ordinary skill in the art would be able to select suitable compounds to inhibit ALK fusion proteins, such as to inhibit EML4-ALK or NPM-ALK.

In some embodiments, the RTK fusion protein is an RET fusion protein. In some embodiments, the RTK fusion protein is CCDC6-RET, KIF5B-RET, NCOA-RET, TRIM33-RET, CUX1-RET, KIAA1468-RET, or combinations thereof. In some embodiments, the inhibitor for the RTK fusion protein is an RET inhibitor. Non-limiting examples of RET inhibitors include alectinib, cabozantinib, lenvatinib, pralsetinib, selpercatinib, sunitinib, or derivatives thereof. RET inhibitors, either specific for RET or having cross-reaction with other RTKs, are well known in the art and one of ordinary skill in the art would be able to select suitable compounds to inhibit RET.

In some embodiments, the MMP inhibitor is a broad-spectrum MMP inhibitor. In some embodiments, the MMP inhibitor is an inhibitor specific for one or more MMPs, such as specific for ADAM 10 or ADAM 17. Non-limiting examples of MMP inhibitors include 1,10-Phenanthroline Monohydrate, Abametapir, Aderbasib, AUDA, Auraptene, Batimastat, Cipemastat, Doxycycline Hyclate, GI254023X, Ilomastat, Lactobionic acid, Ilomastat (Galardin), JNJ0966, Marimastat, MMI270, MMP-9-IN-1, Morroniside, Nobiletin, NSC 405020, o-phenanthroline, Polygalactic acid, Prinomastat, Rebimastat, Ro 28-2653, RS-130830, SB-3CT, Solasodine, Tanomastat, TAPI-1, Trans-Zeatin, Triolein, T-5224, T-26c, or derivatives thereof. Since MMPs are mostly extracellular proteins, in some embodiments, the inhibitor for MMP includes an antibody molecule specific for one or more type of MMPs. MMP inhibitors, either broad-spectrum or specific, are well known in the art and one of ordinary skill in the art would be able to select suitable compounds to inhibit MMPs.

In some embodiments, the subject is a mammal. In some embodiments, the subject is a human.

RTK fusions are prevalent across cancer types and are found in solid tumor cancers and leukemia alike. In some embodiment, the cancer is a lung cancer. In some embodiments, the cancer is a non-small cell lung cancer (NSCLC). In some embodiments, the cancer is an NSCLC driven by EML4-ALK oncogene.

In some embodiments, the subject has previously been treated with an inhibitor for the RTK fusion protein, but not with an inhibitor for MMP. In some embodiment, the cancer in the subject is resistant to a treatment of an inhibitor for the RTK fusion protein alone. In some embodiment, the cancer in the subject is resistant to a treatment of an inhibitor for the RTK fusion protein alone due to a previous treatment with an inhibitor for the RTK fusion protein alone.

### Composition or Kit for Treating or Ameliorating Cancer

As detailed elsewhere herein, the present study establishes that the sequestration of transmembrane RTK signaling pathway effectors, such as Grb2 or an SOS protein (such as SOS1), by an aggregate of an exemplary oncogenic RTK fusion protein (EML4-ALK, which forms a cytoplasmic aggregate) in tumor cells, prevents the effectors from translocating to the membrane to transduce EGF signals. This results in competitive inhibition of the transmembrane RTK signaling. Inhibition of the oncogenic RTK fusion protein alone releases the effectors from the sequestration, thereby re-activating the transmembrane RTK signaling and providing a survival signal in alternative to the ones mediated by the oncogenic RTK fusion protein. This alternative survival signal (at least partially) explains drug resistances that RTK fusion oncogene driven cancers ultimately develop after prolonged treatment with an RTK fusion protein inhibitor.

The present study further establishes that the combined inhibition of the RTK fusion protein (with an inhibitor for the RTK) and the transmembrane RTK signaling pathway (with an inhibitor for MMPs, which cleaves extracellular portions of some transmembrane proteins to produce extracellular EGF signals) achieves a significantly higher death rate in a cancer cell line driven by the RTK fusion oncogene than that achievable by inhibiting the RTK fusion protein alone. Inhibiting the transmembrane RTK signaling pathway alone with the inhibitor for MMP did not result in detectable cell death in the cancer cell line.

As such, the results of the present study illustrate the unexpected results of combining the inhibitor for RTK fusion protein with the inhibitor for MMP protein.

Accordingly, in some aspects, the present invention includes a composition or a kit for treating or ameliorating cancer in a subject in need thereof. In some embodiments, the cancer includes a tumor cell including a receptor tyrosine kinase (RTK) fusion protein aggregation.

In some embodiments, the composition includes an inhibitor for the RTK fusion protein; and an inhibitor for a matrix metalloprotease. In some embodiments, the composition further includes a pharmaceutically acceptable carrier, which is detailed elsewhere herein. In some embodiments, the inhibitor for the RTK fusion protein and the inhibitor for a matrix metalloprotease are co-formulated.

In some embodiments, the kit includes an inhibitor for the RTK fusion protein; and an inhibitor for a matrix metalloprotease. In some embodiments, the kit further includes one or more pharmaceutically acceptable carriers, which is detailed elsewhere herein. In some embodiments, the inhibitor for the RTK fusion protein and the inhibitor for a matrix metalloprotease are formulated separately. In some embodiments, the inhibitor for the RTK fusion protein is formulated with a first pharmaceutically acceptable carrier. In some embodiments, the inhibitor for the MMP is formulated with a second pharmaceutically acceptable carrier.

In some embodiments, the aggregation of the RTK fusion protein sequesters an effector of transmembrane RTK signaling pathway from the transmembrane RTK signaling pathway. In some embodiments, the effector of the transmembrane RTK signaling pathway includes GRB2 or an SOS protein (such as SOS1).

In some embodiments, the RTK fusion protein includes all RTK fusion proteins that form aggregates, membrane or cytoplasmic, in tumor cells. In some embodiments, the RTK fusion protein includes ALK fusions such as EML4-ALK (including all variants thereof), or NPM-ALK; RET fusions such as CCDC6-RET, KIF5B-RET, NCOA-RET, TRIM33-RET, CUX1-RET, or KIAA1468-RET; ROS1 fusions such as TPM3-ROS1, FIG-ROS1, CCDC6-ROS1 or CD74-ROS1; NTRK1 fusions such as MPRIP-NTRK1 or TPM3-NTRK1; and the like. In some embodiments, the aggregation of the RTK fusion protein is a cytoplasmic protein granule. In some embodiments, the RTK fusion protein is a fusion protein including the kinase domain of anaplastic lymphoma kinase (ALK) or the kinase domain of rearranged during transfection (RET). In some embodiments, the fusion protein includes a fusion partner capable of multimerization. Both ALK fusion proteins and RET fusion proteins are known to form cytoplasmic protein granules in tumor cells that include effectors of transmembrane RTKs, such as GRB2 or SOS proteins (e.g., SOS1). As such, it is expected that the effector sequestration as well as the reactivation of transmembrane RTK signaling that causes drug resistance upon inhibiting RTK fusion proteins demonstrated for EML4-ALK is applicable to other ALK fusion proteins and RET fusion proteins. In some embodiments, the fusion protein is at least one selected from the group consisting of EML4-ALK, NPM-ALK, CCDC6-RET, KIF5B-RET, NCOA-RET, TRIM33-RET, CUX1-RET, and KIAA1468-RET.

In some embodiments, the RTK fusion protein is an ALK fusion protein. In some embodiments, the RTK fusion protein is EML4-ALK, NPM-ALK, or a combination thereof. In some embodiments, the inhibitor for the RTK fusion protein is an ALK inhibitor. Non-limiting examples of ALK inhibitors include Alectinib, Alkotinib (also known as ZG-0418), AP26113, ASP3026, AZD3463, Belizatinib (also known as TSR-011), Brigatinib, CEP-28122, CEP-37440, Certinib, Crizotinib, Ensartinib (also known as X-396), Entrectinib (also known as NMS-E628 and RXDX-101), Foritinib (SAF-189), HG-14-10-04, Lorlatinib, PF-06463922, PLB1003, Repotrectinib (also known as TPX-0005), TAE684, TPX-0131, TQ-B3139, TSR-011, X-376, or derivatives thereof. ALK inhibitors are well known and three generations of ALK inhibitors are publicly available. As such, one of ordinary skill in the art would be able to select suitable compounds to inhibit ALK fusion proteins, such as to inhibit EML4-ALK or NPM-ALK.

In some embodiments, the RTK fusion protein is an RET fusion protein. In some embodiments, the RTK fusion protein is CCDC6-RET, KIF5B-RET, NCOA-RET, TRIM33-RET, CUX1-RET, KIAA1468-RET, or combinations thereof. In some embodiments, the inhibitor for the RTK fusion protein is an RET inhibitor. Non-limiting examples of RET inhibitors include alectinib, cabozantinib, lenvatinib, pralsetinib, selpercatinib, sunitinib, or derivatives thereof. RET inhibitors, either specific for RET or having cross-reaction with other RTKs, are well known in the art and one of ordinary skill in the art would be able to select suitable compounds to inhibit RET.

In some embodiments, the MMP inhibitor is a broad-spectrum MMP inhibitor. In some embodiments, the MMP inhibitor is an inhibitor specific for one or more MMPs, such as specific for ADAM 10 or ADAM 17. Non-limiting examples of MMP inhibitors include 1,10-Phenanthroline Monohydrate, Abametapir, AUDA, Aderbasib, Auraptene, Batimastat, Cipemastat, Doxycycline Hyclate, GI254023X, Ilomastat, Lactobionic acid, Ilomastat (Galardin), JNJ0966, Marimastat, MMI270, MMP-9-IN-1, Morroniside, Nobiletin, NSC 405020, o-phenanthroline, Polygalactic acid, Prinomastat, Rebimastat, Ro 28-2653, RS-130830, SB-3CT, Solasodine, Tanomastat, TAPI-1, Trans-Zeatin, Triolein, T-5224, T-26c, or derivatives thereof. Since MMPs are mostly extracellular proteins, in some embodiments, the inhibitor for MMP includes an antibody molecule specific for one or more type of MMPs. MMP inhibitors, either broad-spectrum or specific, are well known in the art and one of ordinary skill in the art would be able to select suitable compounds to inhibit MMPs.

In some embodiments, the subject is a mammal. In some embodiments, the subject is a human.

In some embodiment, the cancer is a lung cancer. In some embodiments, the cancer is a non-small cell lung cancer (NSCLC). In some embodiments, the cancer is an NSCLC driven by EML4-ALK oncogene.

In some embodiments, the subject has previously been treated with an inhibitor for the RTK fusion protein, but not with an inhibitor for MMP. In some embodiment, the cancer in the subject is resistant to a treatment of an inhibitor for the RTK fusion protein alone. In some embodiment, the cancer in the subject is resistant to a treatment of an inhibitor for the RTK fusion protein alone due to a previous treatment with an inhibitor for the RTK fusion protein alone.

### Combination Therapies

In some embodiments, the subject is further administered at least one additional agent that treats, ameliorates, and/or prevents a disease and/or disorder contemplated herein. In other embodiments, the compound and the at least one additional agent are co-administered to the subject, either together, or sequentially, one after the other. In yet other embodiments, the compound and the at least one additional agent are co-formulated.

The compounds contemplated within the disclosure are intended to be useful in combination with one or more additional compounds. These additional compounds may comprise compounds of the present disclosure and/or at least one additional agent for treating cancer, and/or at least one additional agent that treats one or more diseases or disorders contemplated herein.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations contemplated within the disclosure may be administered to the subject either prior to or after the onset of a disease and/or disorder contemplated herein. Further, several divided dosages, as well as staggered dosages may be administered daily or sequentially, or the dose may be continuously infused, or may be a bolus injection. Further, the dosages of the therapeutic formulations contemplated within the disclosure may be proportionally increased or decreased as indicated by the exigencies of the therapeutic or prophylactic situation.

Administration of the compositions contemplated within the disclosure to a patient, preferably a mammal, more preferably a human, may be carried out using known procedures, at dosages and for periods of time effective to treat a disease and/or disorder contemplated herein in the patient. An effective amount of the therapeutic compound necessary to achieve a therapeutic effect may vary according to factors such as the state of the disease or disorder in the patient; the age, sex, and weight of the patient; and the ability of the therapeutic compound contemplated within the disclosure to treat a disease and/or disorder contemplated herein in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound contemplated within the disclosure is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions contemplated within the disclosure may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds contemplated within the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms contemplated within the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease and/or disorder contemplated herein.

In certain embodiments, the compositions of the disclosure are formulated using one or more pharmaceutically acceptable excipients or carriers. In certain embodiments, the pharmaceutical compositions of the disclosure comprise a therapeutically effective amount of a compound of the disclosure and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the disclosure are administered to the patient in dosages that range from one to five times per day or more. In another embodiment, the compositions of the disclosure are administered to the patient in range of dosages that include, but are not limited to, once every day, every two, days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Compounds of the disclosure for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the disclosure is from about 1 mg and about 2,500 mg. In some embodiments, a dose of a compound of the disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of cancer in a patient.

Formulations may be employed in admixtures with conventional excipients, i.e., pharmaceutically acceptable organic or inorganic carrier substances suitable for intracranial, intrathecal, oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the disclosure include oral, nasal, rectal, intravaginal, parenteral, buccal, sublingual or topical. The compounds for use in the disclosure may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the disclosure may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

The present disclosure also includes a multi-layer tablet comprising a layer providing for the delayed release of one or more compounds of the disclosure, and a further layer providing for the immediate release of another medication. Using a wax/pH-sensitive polymer mix, a gastric insoluble composition may be obtained in which the active ingredient is entrapped, ensuring its delayed release.

### Parenteral Administration

For parenteral administration, the compounds of the disclosure may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this disclosure include dosage forms as described in U.S. Pat. Nos. 6,340,475; 6,488,962; 6,451,808; 5,972,389; 5,582,837; and 5,007,790. Additional dosage forms of this disclosure also include dosage forms as described in U.S. Patent Applications Nos. 20030147952; 20030104062; 20030104053; 20030044466; 20030039688; and 20020051820. Additional dosage forms of this disclosure also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present disclosure depends on the age, sex and weight of the patient, the current medical condition of the patient and the progression of the cancer in the patient being treated. The skilled artisan is able to determine appropriate dosages depending on these and other factors.

A suitable dose of a compound of the present disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the modulator of the disclosure is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%,20%,25%,30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the patient's condition, to a level at which the improved disease is retained. In certain embodiments, patients require intermittent treatment on a long-term basis upon any recurrence of symptoms and/or infection.

The compounds for use in the method of the disclosure may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. Capsid assembly modulators exhibiting high therapeutic indices are preferred. The data obtained from cell culture assays and animal studies are optionally used in formulating a range of dosage for use in human. The dosage of such capsid assembly modulators lies preferably within a range of circulating concentrations that include the ED₅₀ with minimal toxicity. The dosage optionally varies within this range depending upon the dosage form employed and the route of administration utilized.

Those skilled in the art recognizes, or is able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in assay and/or reaction conditions, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

### Delivery

In certain embodiments, the compound contemplated herein (including but not limited to nucleic acids) can be more efficiently delivered to the cell nucleus by coupling the compound with the monoclonal anti-DNA antibody 3E10, which penetrates living cells and localizes in the nucleus without causing any apparent harm to the cell (Hansen J E, et al., Intranuclear protein transduction through a nucleoside salvage pathway. J Biol Chem 2007;282:20790-3; see also WO 2020/047353 and WO 2021/042060, all of which are incorporated herein in their entireties by reference). 3E10 and its single-chain variable fragment (3E10 scFv) have been developed as an intracellular delivery system for macromolecules. After localizing in the cell nucleus, 3E10 scFv is largely degraded within 4 hours, thus further minimizing any potential toxicity.

In certain embodiments, the compounds contemplated herein (including but not limited to nucleic acids) can be more efficiently delivered to the cancer using certain lipid nanoparticle formulations known in the art, such as but not limited to those described in Cullis, P. R. et al., Molecular Therapy Vol. 25 No 7 July 2017. See also US20150165039 and WO 2014/008334, all of which are incorporated herein in their entireties by reference.

In certain embodiments, the compounds contemplated herein can be more efficiently delivered to tissue by coupling with certain protein fragments, called “pHLIP” (pH (Low) Insertion Peptide), which allow for the cargo to accumulate in acidic environments within the body. In certain embodiments, a polypeptide with a predominantly hydrophobic sequence long enough to span a membrane lipid bilayer as a transmembrane helix (TM) and comprising one or more dissociable groups inserts across a membrane spontaneously in a pH-dependent fashion placing one terminus inside cell. The polypeptide conjugated with various functional moieties delivers and accumulates them at cell membrane with low extracellular pH. The functional moiety conjugated with polypeptide terminus placed inside cell are translocated through the cell membrane in cytosol. The peptide and its variants or non-peptide analogs can be used to deliver therapeutic, prophylactic, diagnostic, imaging, gene regulation, cell regulation, or immunologic agents to or inside of cells in vitro or in vivo in tissue at low extracellular pH. See also US20080233107, WO2012/021790, US20120039990, US20120142042, US20150051153, US20150086617, and US20150191508, all of which are incorporated herein in their entireties by reference.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1: RTK Signaling is Suppressed in EML4-ALK+ Cancer Cells

A method called functional profiling that allows the measurement of how oncogenes and targeted therapies can alter signal transmission was developed. In functional profiling, light-activated ‘optogenetic’ signaling stimuli are apply at specific nodes of a signaling pathway and downstream signals, transcription, and cell fate as a function of oncogene or drug status are observe.

Here, functional profiling was applied to understand and explore the relationship between transmembrane RTKs and EML4-ALK protein granules as well as its effect on signal transmission.

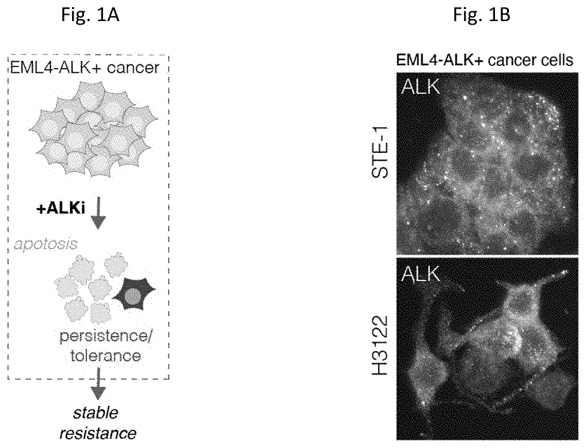

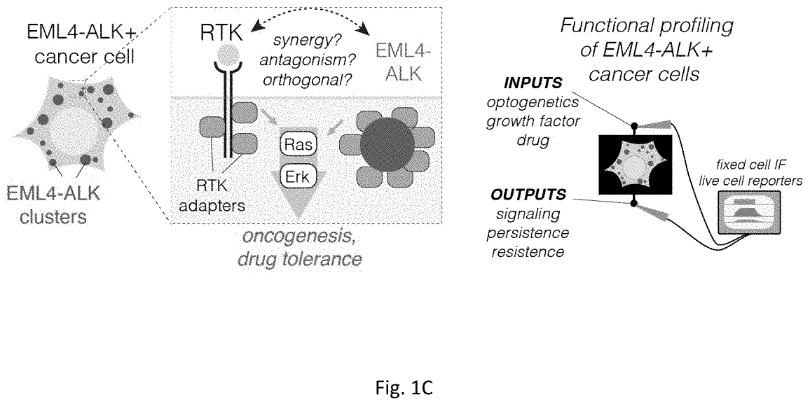

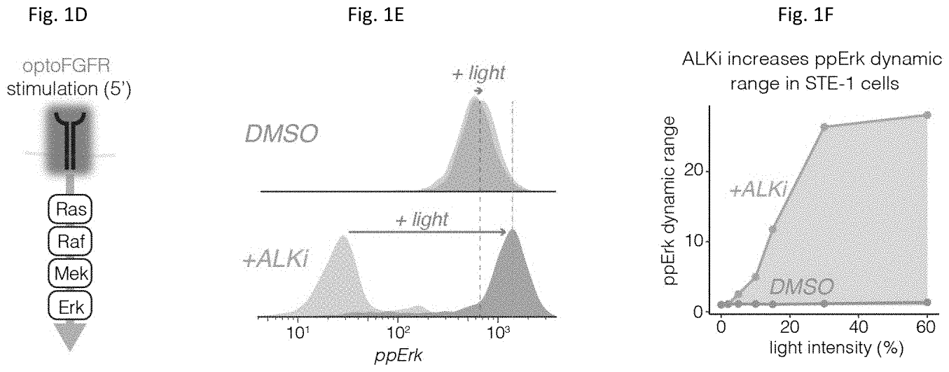

To determine whether EML4-ALK could alter RTK signal transmission properties, a light-sensitive fibroblast growth factor receptor (optoFGFR) was expressed in the STE-1 cancer cell line, which is driven by the EML4-ALK(V1) oncogene. The present study observed signal transmission by applying blue light stimuli and measuring single-cell phospho-Erk levels single-cell immunofluorescence (FIG. 1D). In STE-1 cells, optoFGFR induced only mild double-phosphorylated Erk (ppErk) signal increase above the high basal ppErk levels attributed to active EML4-ALK (FIG. 1E, top). Strikingly, pre-treatment with ALK inhibitor crizotinib (ALKi, 1 μM) dramatically increased the dynamic range of signal induction to —18-fold (FIG. 1E, bottom). Signal dynamic range increased due to lowering of tonic Erk activity from ALK, as well as from an increase in the maximum signal achievable relative to untreated cells. Dynamic range increased as a function of light intensity and plateaued near ˜25-fold induction at the highest light levels of light stimulation. Notably, increase of dynamic range could also be observed with as little as 5% maximal light stimulation, suggesting that ALK inhibition may sensitize cancer cells to exceedingly weak RTK signals (FIG. 1F). It was note that although the magnitude of dynamic range increase was dependent on the expression levels of optoFGFR, the general trends described held across all expression levels. These results suggested the existence of a strong negative regulatory interaction between EML4-ALK expression and transmembrane RTK signaling.

### Example 2: EML4-ALK Suppresses—and ALK Inhibition Restores—EGFR Signaling

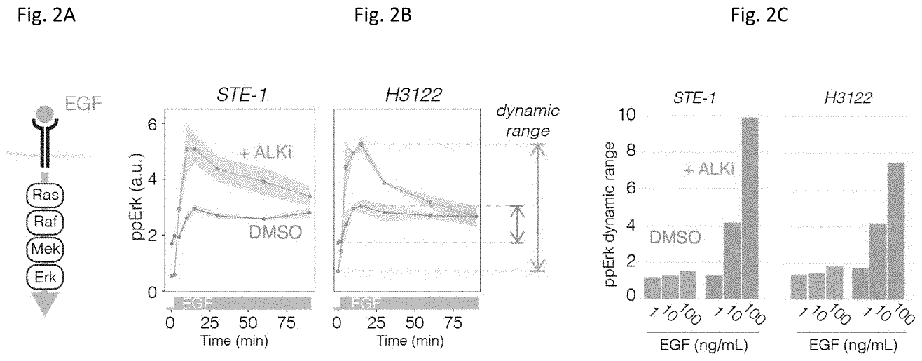

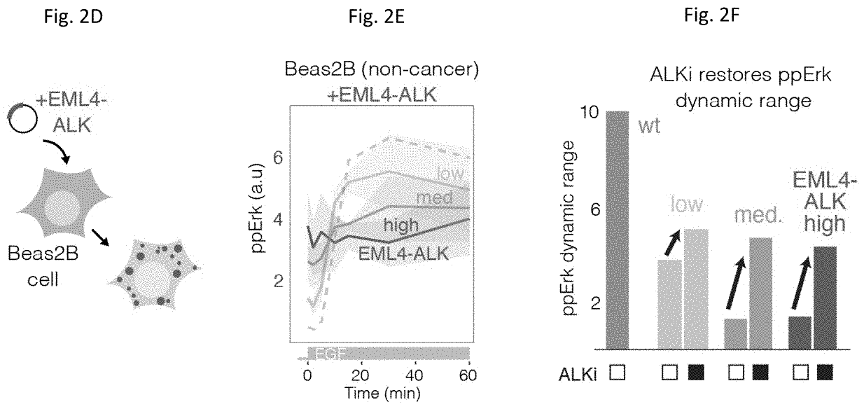

To determine whether such negative regulation could be observed in the context of native epidermal growth factor receptor (EGFR) signaling, the present study measured ppErk induction upon addition of epidermal growth factor (EGF) in STE-1 and H3122 cells, two NSCLC cell lines driven by EML4-ALK(V1) (FIG. 2A). In response to 100 ng/mL EGF, untreated cancer cells showed minimal ppErk signal response. However, pre-treatment with crizotinib resulted in a dramatic increase in ppErk dynamic range in both cell lines, again due to lower baseline—but also higher maximal—ppErk signaling (FIG. 2B-C). Dynamic range increase was observed over at least a 10-fold range of EGF concentrations (FIG. 2C). Thus, it was concluded that EML4-ALK activity negatively regulates the signaling potency of multiple endogenous RTKs in cancer cells. Importantly, dynamic range modulation was also observed in response to the 3rd generation ALK inhibitor lorlatinib but was not observed in cancer cell lines driven by full length ALK. These results indicate that desensitization of transmembrane RTKs was specific to ALK signaling as an RTK fusion, suggesting a role for EML4-ALK aggregation.

The present study then tested whether EML4-ALK(V1) was sufficient for suppression of RTK signaling. An EML4-ALK-2A-miRFP construct was transiently transfected into non-transformed lung epithelial Beas2B cells, and responses to EGF was observed (FIG. 2D). EML4-ALK raised basal ppErk signaling levels, with increased signaling observed with higher EML4-ALK expression (time point 0, FIG. 2E). Upon addition of EGF, the maximal ppErk signal achievable decreased compared to untransfected cells, and suppression of induced signaling increased as a function of EML4-ALK expression (FIG. 2E). Finally, pre-incubation of ALKi reversed EML4-ALK-induced suppression of EGFR signaling (FIG. 2F). Collectively, the results show that EML4-ALK expression is sufficient to desensitize RTK signaling, which can be re-sensitized through ALK inhibition.

### Example 3: EML4-ALK Aggregates Suppress EGFR Through Sequestration of RTK Effectors

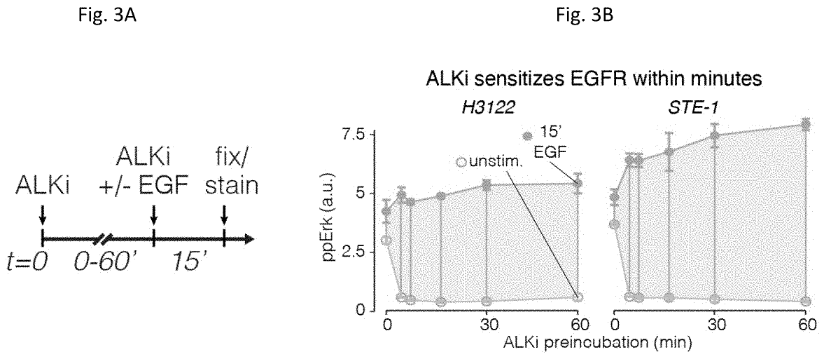

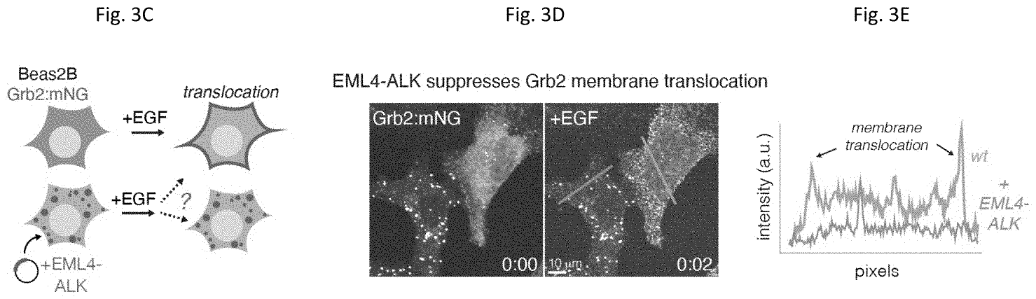

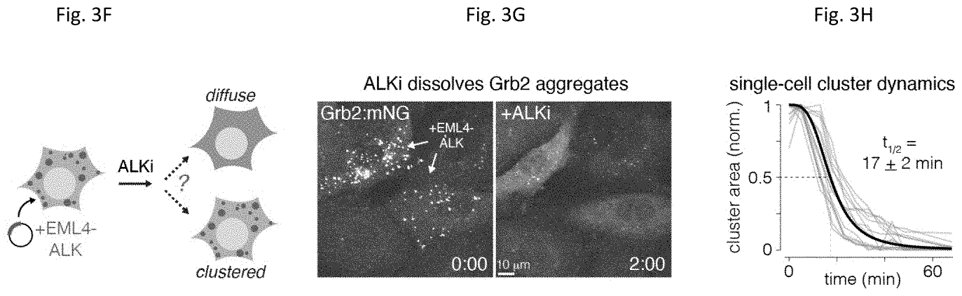

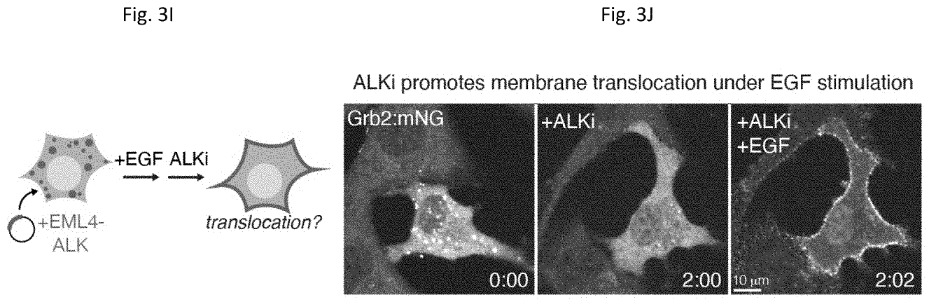

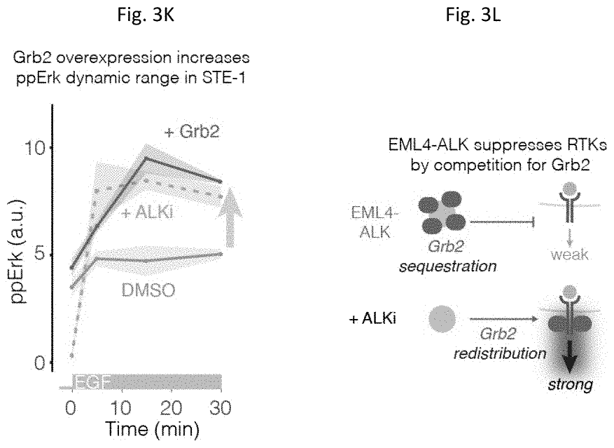

The present study sought to understand the mechanism by which EML4-ALK suppressed EGFR signaling. To determine whether the mechanism was predominantly transcriptional or post-translational, the required duration of ALKi pre-incubation to observe an increase in dynamic range was examined. both STE-1 and H3122 cells were preincubated with varying durations of ALK inhibition, and ppErk levels in response to 15 min of EGF stimulation was analyzed. (FIG. 3A). In both cell lines, EGFR hypersensitization could be observed with as little as 5 min of ALKi pre-incubation (20 minutes total including the 15 min. stimulation) (FIG. 3B). Such fast response indicated a primarily post-translational mechanism.

The present study next sought to understand how EML4-ALK aggregation could suppress EGFR signaling. EML4-ALK aggregates signal from the cell's cytoplasm by recruiting downstream adapters that couple ALK activity to activation of downstream pathways including Ras and phosphatidyl inositol 3 phosphate kinase (PI3K). Since these adapters are required for signaling through both EML4-ALK as well as EGFR, it was hypothesized that the sequestration of these adapters by EML4-ALK would result in competitive inhibition of EGFR. To test this hypothesis, the localization of Grb2, which was fluorescently tagged at the endogenous locus, was observed in Beas2B cells. Grb2 couples phosphorylated EGFR to the Ras-Erk pathway, and is also essential to form and maintain large-scale EML4-ALK aggregates. Upon treatment with 50 ng/mL EGF, Grb2 translocated to the plasma membrane within minutes and appeared to be rapidly endocytosed, presumably with active EGFR receptors (FIGS. 3C-3D). These cells were next transfected with EML4-ALK, which formed large aggregates that recruited Grb2. Upon treatment of transfected cells with EGF, Grb2 remained largely sequestered within aggregates, while Grb2 in neighboring untransfected cells translocated to the membrane (FIGS. 3C-3I). The effect of ALK inhibition on Grb2 distribution were observed next (FIG. 3F). Treatment of transfected cells with crizotinib rapidly dissolved Grb2 clusters, with a half-life of 17+/−2 min (FIGS. 3G-3H). Subsequent EGF addition to crizotinib-treated cells now resulted in robust membrane translocation of Grb2 in EML4-ALK expressing cells (FIGS. 3I-3J). Collectively, the results suggest that EML4-ALK aggregates can functionally sequester Grb2 and prevent Grb2 translocation to the membrane to transduce EGF signals, providing a mechanism by which EML4-ALK can competitively inhibit EGFR signaling.

The imaging results suggest that EML4-ALK suppresses EGFR signaling because of competition for a limited supply of Grb2. This mechanism suggests that increasing the supply of Grb2 would lessen the negative regulation of EML4-ALK on EGFR. To test this prediction, an exogenous fluorescently-tagged Grb2 was expressed in STE-1 cells, and we measured ppErk response to EGF (FIG. 3K). Grb2 expression elevated the maximum EGF-induced ppErk signal, supporting sequestration for Grb2 as a key mechanism of competitive inhibition of EML4-ALK on EGFR (FIG. 3L).

### Example 4: RTK Hypersensitization Upon ALK Inhibition results in Rapid Signal Reactivation Through Paracrine Signaling

The present study sought to understand whether relief of EGFR inhibition by EML4-ALK might lead to Erk reactivation in STE-1 and H3122 cancer cells. The present study first examined single-cell ppErk levels as a function of crizotinib treatment duration. Upon treatment, mean Erk levels decreased dramatically and rapidly, within the first 10 minutes. However, within ˜1 hr, a small population of cells having high level of ppERK (ERK-hi cells) reappeared. Co-treatment with crizotinib and EGFR inhibitor erlotinib prevented reappearance of the ERK-hi population, suggesting that reappearance of ERK-hi cells depended on EGFR signaling. Since the reappearance of ERK-hi cells happened under serum-starved conditions, it was hypothesized that ERK-hi cells resulted from paracrine signaling within the cancer cell population. The present study thus further examined Erk reactivation under ALK inhibition combined with marimastat, an inhibitor for matrix metalloproteases that are required for shedding EGF family ligands prior to paracrine signaling. Cotreatment with marimastat reduced the number of cells that showed Erk reactivation, consistent with reactivation due to paracrine signaling.

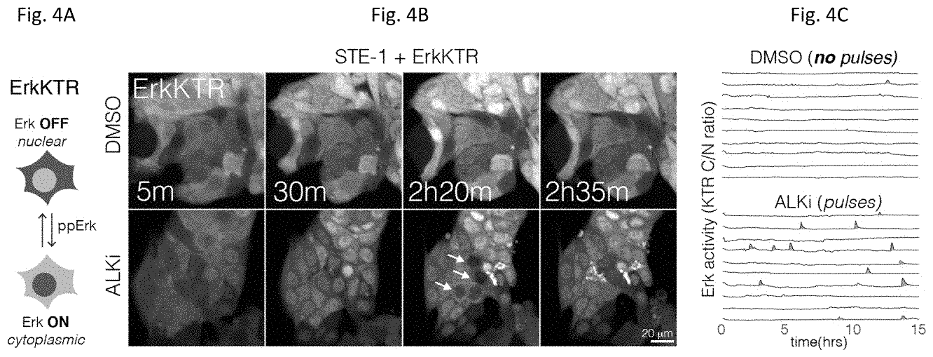

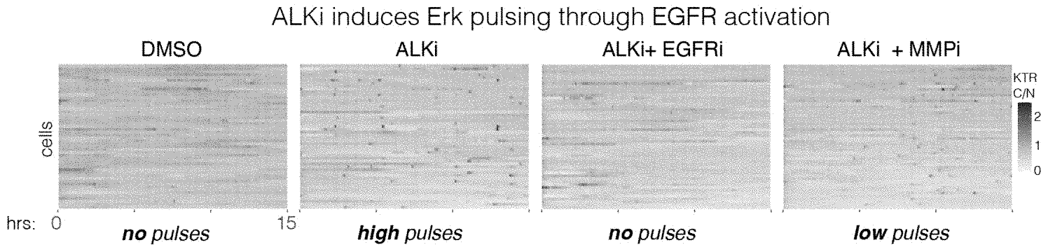

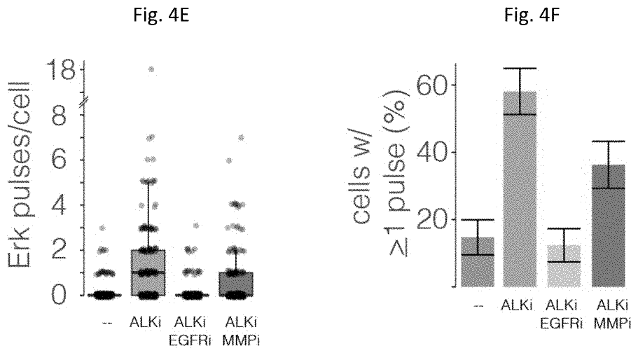

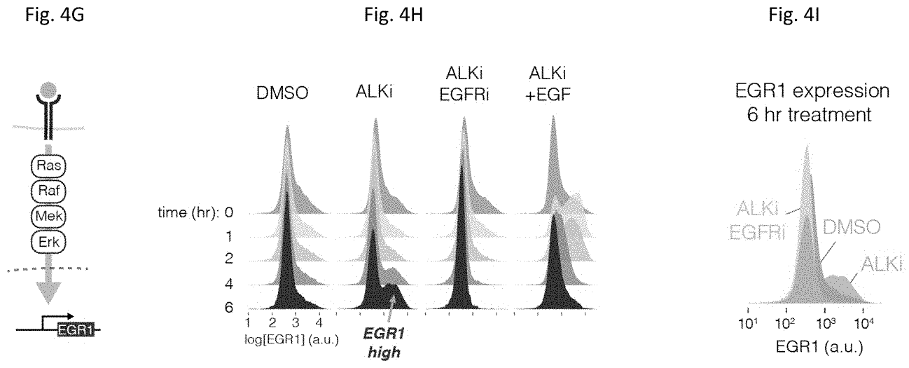

To further understand Erk reactivation, the present study observed Erk activity using live cell imaging of the Erk kinase translocation reporter (ErkKTR) in STE-1 cells (FIG. 4A). ErkKTR is a fluorescence reporter whose localization corresponds with Erk activity: the nuclear localization of ErkKTR indicates low activity of Erk while the cytoplasmic localization indicates high activity of Erk. In the absence of ALK inhibitor, ErkKTR was largely cytoplasmic, reflecting intermediate tonic Erk activity (FIG. 4B). Treatment with crizotinib induced rapid nuclear translocation of ErkKTR in all cells, in agreement with rapid Erk dephosphorylation. However, within as little as 1 hr, Erk activity began pulsing with ˜15 min durations in individual cells. The amplitude of ErkKTR activity (nuclear/cytoplasm ratio) surpassed the amplitude of reporter activity in the absence of drug (FIGS. 4B-4C). Neither signal decrease nor pulsatile Erk were observed in cells treated with DMSO. In cells co-treated with crizotinib and erlotinib, Erk activity decreased but effectively no reactivation or pulsatile Erk activity was observed, showing that Erk reactivation requires EGFR signals (FIGS. 4D-4F). Finally, co-treatment of crizotinib with marimastat reduced both basal Erk activity and Erk reactivation pulses, though a small number of Erk reactivation pulses could still be observed (FIGS. 4D-4F). Although treatment with crizotinib decreased Erk activity and then resulted in EGFR-dependent activity pulses, treatment with only erlotinib or only marimastat had no obvious effect on ErkKTR, suggesting that cells with intact signaling did not respond to environmental RTK signals.

To understand the extent to which such rapid and sporadic Erk activity pulses could impact cell behavior, the present study asked whether the Erk pulses could stimulate downstream transcription. Erk activity is a strong driver of transcription, including of a class of rapidly responding genes called immediate early genes. EGR1 is a transcription factor in the IEG class that is strongly expressed but then rapidly adapts in response to an Erk stimulus. Thus, accumulation of EGR1 would indicate the presence of recent Erk activity. The present study thus examined the extent to which EGR1 accumulated in STE-1 cells upon drug treatment (FIG. 4G). In untreated cancer cells, EGR1 levels remained mostly low, likely due to the tonic Erk signaling resulting from EML4-ALK expression. Upon ALK inhibition, EGR1 levels remained low for at least 2 hrs, but a distinct peak of EGR1-high cells appeared and grew at 4 and 6 hrs after drug treatment, indicating that ALKi-induced Erk pulses were indeed driving transcription in STE-1 cells (FIG. 4H). Importantly, co-inhibition of ALK and EGFR completely eliminated the EGR1-positive population, demonstrating that EGR1 expression was being driven through EGFR stimulation, likely through paracrine signaling (FIGS. 4H-4I).

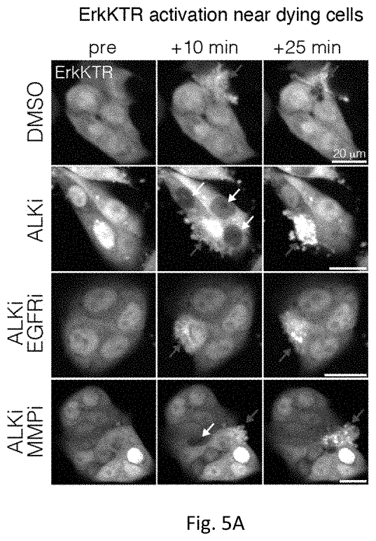

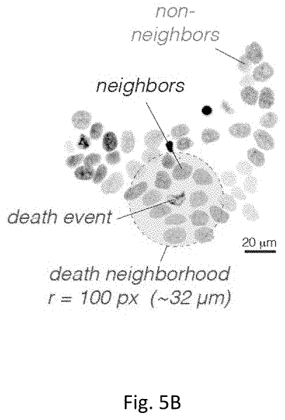

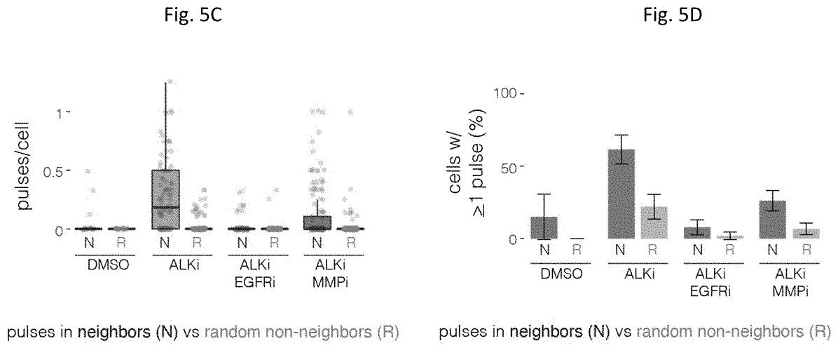

The present study next sought to determine the source of the paracrine signals. Inspection of the live-cell ErkKTR imaging provided several clues: 1) Erk activation often propagated across neighboring cells within a colony, 2) activation was frequently observed near and preceding a cell death event, and 3) co-treatment of EGFRi or MMPi appeared to eliminate signal activation in cells neighboring to dying cells (FIG. 5A). These observations suggest paracrine signal secretion from dying cells, which promote cell survival and homeostasis within epithelial sheets. Signal activation near dying cells was quantified by first identifying the time and location of each cell death event. All cells that were within a 100-pixel radius (˜32 μm, or about a 2-cell radius) of the dying cell for the 50 minutes preceding death were then identified. The present study refers to these as the “neighbors” of the dying cell (FIG. 5B). The present study then counted Erk pulses in the neighbors of a death event and compared to Erk pulses from a randomly selected subset of non-neighbors over that same time interval. The analysis showed that pulses were not observed in the large majority of untreated cells. However, in cells treated with ALKi, the neighbors of a death showed a median of 0.23 pulses per cell, compared to 0 for randomly selected non-neighbors (FIG. 5C). Co-treatment with EGFRi eliminated Erk pulsing in neighbors, while co-treatment with MMPi dramatically reduced neighbor signaling (FIG. 5C). From these neighbor/non-neighbor populations, the proportion of cells that showed at least 1 pulse preceding a death event was then quantified. The present study found significantly more pulsing in neighbor cells vs non-neighbors in ALKi treated cells (60% vs 20%, significant via non-overlapping 95% confidence intervals) and ALKi/MMPi co-treated cells (30% vs 10%) (FIG. 5D). No differences were found between neighbor and non-neighbor populations in DMSO or ALKi/EGFRi co-treated cells. Together, the results confirm that most Erk signaling induced in STE-1 cells upon ALKi treatment results from paracrine signaling

Collectively, the results show that EML4-ALK expression renders cancer cells insensitive to RTK signals in their environment. ALK inhibition lowers tonic oncogenic signaling, but simultaneously sensitizes cells to strong RTK pathway reactivation through signals from neighboring cells.

### Example 5: Signal Reactivation Promotes Acute Drug Tolerance and Cell Persistence in the Presence of ALK Inhibition

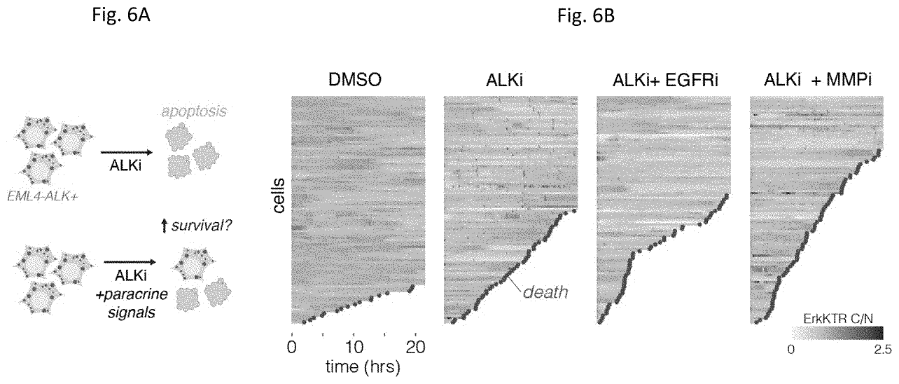

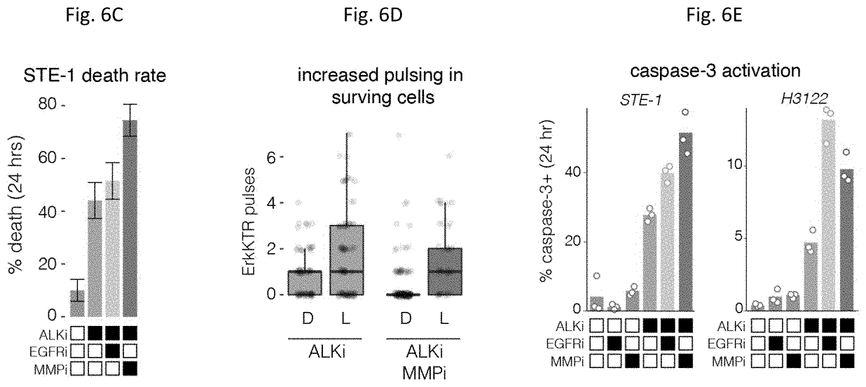

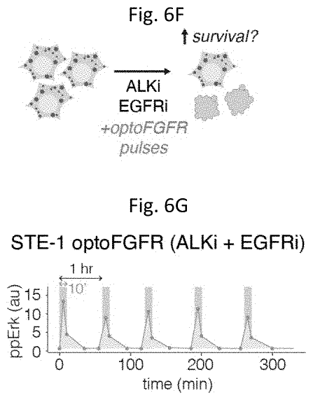

Signal reactivation has been implicated as a central route of drug tolerance and resistance development in EML4-ALK positive cells, and death-induced paracrine signaling can promote survival in neighboring cells in epithelial sheets. The present study thus asked whether the observed signal reactivation could promote survival during ALK inhibitor treatment (FIG. 6A). In STE-1 cells imaged over 24 hrs, ALKi treatment induced death in ˜40% of cells analyzed. However, concurrent blockade of EGFR or MMPs increased cell death to 50 and 70% of cells, respectively (FIGS. 6B-6C). In both ALKi- and ALKi/MMPi-treated cells, surviving cells showed an increased number of Erk pulses compared to cells that died, further drawing a link between RTK signaling and cell survival (FIG. 6D). To confirm the results through a separate assay, the present study examined activation of a fluorescent reporter of caspase-3 activity (Nucview), an indicator of apoptosis, through the first 24 hr of treatment in both STE-1 and H3122 cell lines. Here again, while ALK inhibition led to increased caspase-3, co-treatment with EGFRi increased the caspase-3-positive cell fraction (from 25 to 40% and from 5 to 13% in STE-1 and H3122, respectively). Similarly, co-treatment with MMPi also increased caspase-3 (from 25 to 50% in STE-1 and from 5 to 10% in H3122) (FIG. 6E) Neither erlotinib or marimastat treatment alone showed enhanced killing over untreated cells.

Together, the results suggest that rapid RTK signal reactivation resulting from relief of RTK inhibition and paracrine signals limits the cytotoxicity of ALK therapies, suggesting novel co-treatment targets for EML4-ALK+ cancers.

### Example 6

The present study demonstrates that protein granules of oncogenic EML4-ALK antagonize the function of transmembrane EGFR signaling through a competitive inhibition for the downstream adapter Grb2, and that this competitive inhibition relies on sequestration through EML4-ALK aggregates. ALK inhibition, though effective in reducing tonic phospho-Erk levels and promoting tumor regression, releases Grb2 sequestration by EML4-ALK, relieves suppression of EGFR signaling, and sensitizes cells to environmental EGFR ligands that counteract therapeutic ALK responses.

Despite successful approval of three generations of ALK inhibitors for NSCLC, acquired resistance to ALK inhibitors emerges with high frequency, demonstrating an urgent need to understand and counteract the mechanisms that underlie resistance development.

The present study suggests new strategies. The present study discovered that marimastat is able to significantly enhance killing under ALK inhibition, which suggests that inhibitors of metalloproteases could enhance cell killing and prevent signal reactivation that drives tolerance and drug resistance. The present study further suggests that new strategies that prevent Grb2 redistribution or that mimic EML4-ALK and sequester Grb2 could prevent significant RTK signaling and drug resistance. Notably, the recent discovery that EML4-ALK signals from aggregates has motivated a search for molecules that disaggregate or otherwise disrupt the biophysical assemblies required for ALK signal transduction. By contrast, the present study predicts that such EML4-ALK disaggregation strategies will be subject to the same rapid RTK reactivation observed with ALK inhibitors because of RTK potentiation due to Grb2 redistribution.

The present study ascribes therapeutically important roles to the EML4-ALK aggregation that functionally link transmembrane RTKs and cytoplasmic EML4-ALK. First, aggregates act as a sink for RTK adapters, which functionally desensitizes transmembrane RTKs to environmental signals. Second, aggregates implement a buffer that rapidly hypersensitizes RTK signaling after ALK inhibition, promoting drug tolerance and cell survival. The results thus advance novel roles for how higher-order protein assemblies can contribute to cancer and its response to therapy.

Because the suppression of EGFR by EML4-ALK results from clustering of the ALK domain, and because multimerization is a common feature of RTK fusions and higher order aggregation may be a common feature of RTK fusions, it is expected that the findings of the present study are broadly applicable to other fusion-positive cancer cells.

Indeed, the present study further shows that, in a cancer line driven by a CCDC6-RET fusion that also forms large aggregates similar to EML4-ALK, RET activity similarly suppresses EGFR, and RET inhibition potentiates EGFR signaling. Furthermore, Grb2 changes localization from the RTK fusion to EGFR under inhibition of the fusion in cancer lines driven by EML4-ALK, or fusions of RET, NTRK1, and ROS1 (Vaishnavi et al., Cancer Res. (77) (13) 3551-3563 (2017)). These results demonstrate the potentially widespread prevalence of competitive inhibition of EGFR by RTK fusions. Therefore, it is expected that such inhibition is observed not only on EGFR but across a large set of RTKs, since suppression was also observed using optogenetic FGFR stimulation.

### Example 7: Synergistic Anti-Cancer Effects of ALK Inhibitor and MMP Inhibitor are Not Limited by ALK Inhibitor Types

To exclude the unlikely possibility that the synergistic anti-cancer effects of ALK inhibitor and MMP inhibitor described above are limited to the ALK inhibitor, crizotinib (a first generation ALK inhibitor), the present study tested the anti-cancer effect of the combination of another ALK inhibitor, alectinib (a second generation ALK inhibitor), and MMP inhibitors.

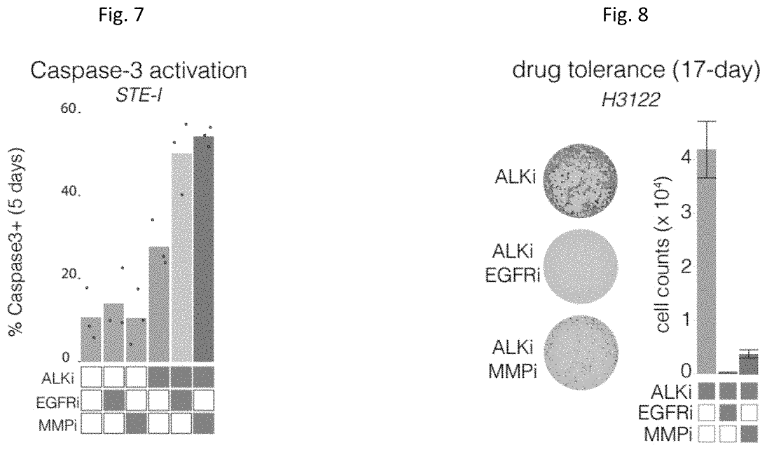

Referring to FIG. 7, the second generation ALK inhibitor alectinib also displayed synergistic anti-cancer effects with the MMP inhibitor marimastat, as the combination of the two inhibitors was significantly more effective at killing STE-1 cancer cells relative to either single agent alone.

### Example 8: Combination of ALK Inhibitor and MMP Inhibitor is Able to Achieve Longer Term Cancer-Killing Effects

Referring to FIG. 8, the combination therapy using both an ALK inhibitor and an MMP inhibitor was not only effective in killing cells faster in 1-4 day experiments, but also was effective over longer time courses. Specifically, FIG. 8 shows that, at 17 days of treatment, while single-agent crizotinib treated H3122 cancer cells started to grow out again, the crizotinib/MMPi co-treatment cells did not.

### Enumerated Embodiments

In some aspects, the present invention is directed to the following non-limiting embodiments:

Embodiment 1: A method of treating or ameliorating a cancer in a subject in need thereof, comprising administering to the subject:
an effective amount of an inhibitor for the receptor tyrosine kinase (RTK) fusion protein; and
an effective amount of an inhibitor for a matrix metalloprotease (MMP),
wherein a tumor cell in the cancer comprises an aggregate of the RTK fusion protein.

Embodiment 2: The method of Embodiment 1, wherein the RTK fusion protein is at least one selected from the group consisting of an anaplastic lymphoma kinase (ALK) fusion protein, a rearranged during transfection (RET) fusion protein, a ROS1 fusion protein, and a neurotrophic receptor tyrosine kinase 1 (NTRK1) fusion protein.

Embodiment 3: The method of any of Embodiments 1-2, wherein the RTK fusion protein is at least one selected from the group consisting of EML4-ALK, NPM-ALK, CCDC6-RET, KIF5B-RET, NCOA-RET, TRIM33-RET, CUX1-RET, and KIAA1468-RET.

Embodiment 4: The method of any of Embodiments 1-3, wherein the cytoplasmic granule of the RTK fusion protein sequesters an effector of a transmembrane RTK signaling pathway from the transmembrane RTK signaling pathway.

Embodiment 5: The method of any of Embodiments 1-4, wherein the cytoplasmic granule of the RTK fusion protein sequesters growth factor receptor bound protein 2 (GRB2) or a son of sevenless (SOS) protein from a transmembrane RTK signaling pathway.

Embodiment 6: The method of any of Embodiments 1-5, wherein the RTK fusion protein comprises an ALK fusion protein, and wherein the inhibitor for the RTK fusion protein comprises at least one ALK inhibitor selected from the group consisting of Alectinib, Alkotinib (also known as ZG-0418), AP26113, ASP3026, AZD3463, Belizatinib (also known as TSR-011), Brigatinib, CEP-28122, CEP-37440, Certinib, Crizotinib, Ensartinib (also known as X-396), Entrectinib (also known as NMS-E628 and RXDX-101), Foritinib (SAF-189), HG-14-10-04, Lorlatinib, PF-06463922, PLB1003, Repotrectinib (also known as TPX-0005), TAE684, TPX-0131, TQ-B3139, TSR-011, X-376, and a derivative thereof.

Embodiment 7: The method of any of Embodiments 1-6, wherein the RTK fusion protein comprises an RET fusion protein, and wherein the inhibitor for the RTK fusion protein comprises at least one selected from the group consisting of alectinib, cabozantinib, lenvatinib, pralsetinib, selpercatinib, sunitinib, and a derivative thereof.

Embodiment 8: The method of any of Embodiments 1-7, wherein the MMP comprises at least one selected from the group consisting of ADAM 10 and ADAM 17.

Embodiment 9: The method of any of Embodiments 1-8, wherein the inhibitor for MMP comprises at least one selected from the group consisting of 1,10-Phenanthroline Monohydrate, Abametapir, Aderbasib, AUDA, Auraptene, Batimastat, Cipemastat, Doxycycline Hyclate, GI254023X, Ilomastat, Lactobionic acid, Ilomastat (Galardin), JNJ0966, Marimastat, MMI270, MMP-9-IN-1, Morroniside, Nobiletin, NSC 405020, o-phenanthroline, Polygalactic acid, Prinomastat, Rebimastat, Ro 28-2653, RS-130830, SB-3CT, Solasodine, Tanomastat, TAPI-1, Trans-Zeatin, Triolein, T-5224, T-26c, and a derivative thereof.

Embodiment 10: The method of any of Embodiments 1-9, wherein the subject is a human.

Embodiment 11: A pharmaceutical composition or kit for treating or ameliorating a cancer in a subject in need thereof, comprising:
an inhibitor for the RTK fusion protein;
an inhibitor for a matrix metalloprotease (MMP); and
at least one pharmaceutically acceptable carrier,
wherein a tumor cell in the cancer comprises an aggregate of a receptor tyrosine kinase (RTK) fusion protein.

Embodiment 12: The pharmaceutical composition or kit of Embodiment 11, wherein the RTK fusion protein is at least one selected from the group consisting of an anaplastic lymphoma kinase (ALK) fusion protein, a rearranged during transfection (RET) fusion protein, a ROS1 fusion protein, and a neurotrophic receptor tyrosine kinase 1 (NTRK1) fusion protein.

Embodiment 13: The pharmaceutical composition or kit of any of Embodiments 11-12, wherein the RTK fusion protein is at least one selected from the group consisting of EML4-ALK, NPM-ALK, CCDC6-RET, KIF5B-RET, NCOA-RET, TRIM33-RET, CUX1-RET, and KIAA1468-RET.

Embodiment 14: The pharmaceutical composition or kit of any of Embodiments 11-13, wherein the cytoplasmic granule of the RTK fusion protein sequesters an effector of a transmembrane RTK signaling pathway from the transmembrane RTK signaling pathway.

Embodiment 15: The pharmaceutical composition or kit of any of Embodiments 11-14, wherein the cytoplasmic granule of the RTK fusion protein sequesters growth factor receptor bound protein 2 (GRB2) or a son of sevenless (SOS) protein from a transmembrane RTK signaling pathway.

Embodiment 16: The pharmaceutical composition or kit of any of Embodiments 11-15, wherein the RTK fusion protein comprises an ALK fusion protein, and wherein the inhibitor for the RTK fusion protein comprises at least one ALK inhibitor selected from the group consisting of Alectinib, Alkotinib (also known as ZG-0418), AP26113, ASP3026, AZD3463, Belizatinib (also known as TSR-011), Brigatinib, CEP-28122, CEP-37440, Certinib, Crizotinib, Ensartinib (also known as X-396), Entrectinib (also known as NMS-E628 and RXDX-101), Foritinib (SAF-189), HG-14-10-04, Lorlatinib, PF-06463922, PLB1003, Repotrectinib (also known as TPX-0005), TAE684, TPX-0131, TQ-B3139, TSR-011, X-376, and a derivative thereof.

Embodiment 17: The pharmaceutical composition or kit of any of Embodiments 11-16, wherein the RTK fusion protein comprises an RET fusion protein, and wherein the inhibitor for the RTK fusion protein comprises at least one selected from the group consisting of alectinib, cabozantinib, lenvatinib, pralsetinib, selpercatinib, sunitinib, and a derivative thereof.

Embodiment 18: The pharmaceutical composition or kit of any of Embodiments 11-17, wherein the MMP comprises at least one selected from the group consisting of ADAM 10 and ADAM 17.

Embodiment 19: The pharmaceutical composition or kit of any of Embodiments 11-18, wherein the inhibitor for MMP comprises at least one selected from the group consisting of 1,10-Phenanthroline Monohydrate, Abametapir, Aderbasib, AUDA, Auraptene, Batimastat, Cipemastat, Doxycycline Hyclate, GI254023X, Ilomastat, Lactobionic acid, Ilomastat (Galardin), JNJ0966, Marimastat, MMI270, MMP-9-IN-1, Morroniside, Nobiletin, NSC 405020, o-phenanthroline, Polygalactic acid, Prinomastat, Rebimastat, Ro 28-2653, RS-130830, SB-3CT, Solasodine, Tanomastat, TAPI-1, Trans-Zeatin, Triolein, T-5224, T-26c, and a derivative thereof.

Embodiment 20: The pharmaceutical composition or kit of any of Embodiments 11-19, wherein the subject is a human.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A method of treating or ameliorating a cancer in a subject in need thereof, comprising administering to the subject:
an effective amount of an inhibitor for the receptor tyrosine kinase (RTK) fusion protein; and
an effective amount of an inhibitor for a matrix metalloprotease (MMP),
wherein a tumor cell in the cancer comprises an aggregate of the RTK fusion protein.

2. The method of claim 1, wherein the RTK fusion protein is at least one selected from the group consisting of an anaplastic lymphoma kinase (ALK) fusion protein, a rearranged during transfection (RET) fusion protein, a ROS1 fusion protein, and a neurotrophic receptor tyrosine kinase 1 (NTRK1) fusion protein.

3. The method of claim 1, wherein the RTK fusion protein is at least one selected from the group consisting of EML4-ALK, NPM-ALK, CCDC6-RET, KIF5B-RET, NCOA-RET, TRIM33-RET, CUX1-RET, KIAA1468-RET, TPM3-ROS1, FIG-ROS1, CCDC6-ROS1, CD74-ROS1, MPRIP-NTRK1, and TPM3-NTRK1.

4. The method of claim 1, wherein a cytoplasmic granule of the RTK fusion protein sequesters an effector of a transmembrane RTK signaling pathway from the transmembrane RTK signaling pathway.

5. The method of claim 1, wherein a cytoplasmic granule of the RTK fusion protein sequesters growth factor receptor bound protein 2 (GRB2) or a son of sevenless (SOS) protein from a transmembrane RTK signaling pathway.

6. The method of claim 1, wherein the RTK fusion protein comprises an ALK fusion protein, and wherein the inhibitor for the RTK fusion protein comprises at least one ALK inhibitor selected from the group consisting of Alectinib, Alkotinib (also known as ZG-0418), AP26113, ASP3026, AZD3463, Belizatinib (also known as TSR-011), Brigatinib, CEP-28122, CEP-37440, Certinib, Crizotinib, Ensartinib (also known as X-396), Entrectinib (also known as NMS-E628 and RXDX-101), Foritinib (SAF-189), HG-14-10-04, Lorlatinib, PF-06463922, PLB1003, Repotrectinib (also known as TPX-0005), TAE684, TPX-0131, TQ-B3139, TSR-011, X-376, and a derivative thereof.

7. The method of claim 1, wherein the RTK fusion protein comprises an RET fusion protein, and wherein the inhibitor for the RTK fusion protein comprises at least one selected from the group consisting of alectinib, cabozantinib, lenvatinib, pralsetinib, selpercatinib, sunitinib, and a derivative thereof.

8. The method of claim 1, wherein the MMP comprises at least one selected from the group consisting of ADAM 10 and ADAM 17.

9. The method of claim 1, wherein the inhibitor for MMP comprises at least one selected from the group consisting of 1,10-Phenanthroline Monohydrate, Abametapir, Aderbasib, AUDA, Auraptene, Batimastat, Cipemastat, Doxycycline Hyclate, GI254023X, Ilomastat, Lactobionic acid, Ilomastat (Galardin), JNJ0966, Marimastat, MMI270, MMP-9-IN-1, Morroniside, Nobiletin, NSC 405020, o-phenanthroline, Polygalactic acid, Prinomastat, Rebimastat, Ro 28-2653, RS-130830, SB-3CT, Solasodine, Tanomastat, TAPI-1, Trans-Zeatin, Triolein, T-5224, T-26c, and a derivative thereof.

10. The method of claim 1, wherein the subject is a human.

11. A pharmaceutical composition or a kit for treating or ameliorating a cancer in a subject in need thereof, comprising:
an inhibitor for the RTK fusion protein;
an inhibitor for a matrix metalloprotease (MMP); and
at least one pharmaceutically acceptable carrier,
wherein a tumor cell in the cancer comprises an aggregate of a receptor tyrosine kinase (RTK) fusion protein.

12. The pharmaceutical composition or kit of claim 11, wherein the RTK fusion protein is at least one selected from the group consisting of an anaplastic lymphoma kinase (ALK) fusion protein, a rearranged during transfection (RET) fusion protein, a ROS1 fusion protein, and a neurotrophic receptor tyrosine kinase 1 (NTRK1) fusion protein.

13. The pharmaceutical composition or kit of claim 11, wherein the RTK fusion protein is at least one selected from the group consisting of EML4-ALK, NPM-ALK, CCDC6-RET, KIF5B-RET, NCOA-RET, TRIM33-RET, CUX1-RET, KIAA1468-RET, TPM3-ROS1, FIG-ROS1, CCDC6-ROS1, CD74-ROS1, MPRIP-NTRK1, and TPM3-NTRK1.

14. The pharmaceutical composition or kit of claim 11, wherein a cytoplasmic granule of the RTK fusion protein sequesters an effector of a transmembrane RTK signaling pathway from the transmembrane RTK signaling pathway.

15. The pharmaceutical composition or kit of claim 11, wherein a cytoplasmic granule of the RTK fusion protein sequesters growth factor receptor bound protein 2 (GRB2) or a son of sevenless (SOS) protein from a transmembrane RTK signaling pathway.

16. The pharmaceutical composition or kit of claim 11, wherein the RTK fusion protein comprises an ALK fusion protein, and wherein the inhibitor for the RTK fusion protein comprises at least one ALK inhibitor selected from the group consisting of Alectinib, Alkotinib (also known as ZG-0418), AP26113, ASP3026, AZD3463, Belizatinib (also known as TSR-011), Brigatinib, CEP-28122, CEP-37440, Certinib, Crizotinib, Ensartinib (also known as X-396), Entrectinib (also known as NMS-E628 and RXDX-101), Foritinib (SAF-189), HG-14-10-04, Lorlatinib, PF-06463922, PLB1003, Repotrectinib (also known as TPX-0005), TAE684, TPX-0131, TQ-B3139, TSR-011, X-376, and a derivative thereof.

17. The pharmaceutical composition or kit of claim 11, wherein the RTK fusion protein comprises an RET fusion protein, and wherein the inhibitor for the RTK fusion protein comprises at least one selected from the group consisting of alectinib, cabozantinib, lenvatinib, pralsetinib, selpercatinib, sunitinib, and a derivative thereof.

18. The pharmaceutical composition or kit of claim 11, wherein the MMP comprises at least one selected from the group consisting of ADAM 10 and ADAM 17.

19. The pharmaceutical composition or kit of claim 11, wherein the inhibitor for MMP comprises at least one selected from the group consisting of 1,10-Phenanthroline Monohydrate, Abametapir, Aderbasib, AUDA, Auraptene, Batimastat, Cipemastat, Doxycycline Hyclate, GI254023X, Ilomastat, Lactobionic acid, Ilomastat (Galardin), JNJ0966, Marimastat, MMI270, MMP-9-IN-1, Morroniside, Nobiletin, NSC 405020, o-phenanthroline, Polygalactic acid, Prinomastat, Rebimastat, Ro 28-2653, RS-130830, SB-3CT, Solasodine, Tanomastat, TAPI-1, Trans-Zeatin, Triolein, T-5224, T-26c, and a derivative thereof.

20. The pharmaceutical composition or kit of claim 11, wherein the subject is a human.
